(19) United States
(12) Patent Application Publication
(10) Pub. No.: US 2023/0270083 A1
(43) Pub. Date: Aug. 31, 2023

(54) WATER TANK MONITORING

(71) Applicant: BLUSTREAM CORPORATION, Worcester, MA (US)

(72) Inventors: Michael Dominick Audi, Worcester, MA (US); William Buote, Worcester, MA (US); Kenneth N. Rapp, Sutton, MA (US); Robert Bean, Worcester, MA (US); James Michael Gordon, JR., Worcester, MA (US)

(21) Appl. No.: 18/040,726

(22) PCT Filed: Aug. 6, 2021
(86) PCT No.: PCT/US2021/045112

§ 371 Date: Feb. 6, 2023

Related U.S. Application Data
(60) Provisional application No. 63/062,825, filed on Aug. 7, 2020.

(51) Int. Cl. A01K 63/04; G01N 33/18; G01N 27/06; G01F 23/72; A01K 63/06; A01K 61/85

(52) U.S. Cl. CPC A01K 63/04; G01N 33/18; G01N 27/06; G01F 23/72; A01K 63/065; A01K 61/85

(57) ABSTRACT

A method of monitoring water tank includes receiving a temperature data set, a water conductivity data set, and a water level data set, characterizing measurements of a plurality of characteristic properties of water within a water tank by a sensor operatively arranged within the water tank. The method further includes determining a first rule characterizing environmental requirements of the water arranged within the water tank. The method further includes generating an output data set based on the received data sets and the first rule. The method further includes providing the generated output data set. Related apparatus, systems, techniques, and articles are also described.

### RELATED APPLICATION

This application claims priority under 35 U.S.C. § 119(e) to U.S. Provisional Patent Application No. 63/062,825 filed on Aug. 7, 2020, the entire contents of which is hereby expressly incorporated by reference herein.

### TECHNICAL FIELD

The subject matter described herein relates to monitoring water quality in tanks for living aquatic species.

### BACKGROUND

Water tanks for aquatic species can require certain inputs in order to keep the environment stable. Sensors can be a part of determining the factors that influence the water tank. A sensor can detect physical properties such as motion, temperature, position, and the like, and generate a signal that represents the detected physical property. The generated signal can be processed and stored by a computing device. The computing device can control the operation of the sensor. For example, the computing device can control the operation of the sensor (e.g., change the operating parameters associated with the sensor).

Water tanks are commonly used for recreational and commercial purposes to keep fish and aquatic animals. The condition of the water tanks (e.g., pH value, ammonia content, etc.) may need to be maintained at desirable levels (e.g., to mimic the natural environmental conditions of the aquatic animals in the tank). In order to maintain the health of the aquatic animals, the water in the tank may need to be regularly treated, filtered and exchanged. Maintenance operations can be performed based on manual measurements (e.g., occasional, infrequent, etc.) of water conditions or on periodic schedules based on historical norms or averages that have been observed to be appropriate and conservative. However, these schedules do not provide a response to the actual conditions in the tank (e.g., in real-time), and may not account for an accelerated deterioration. Alternately or additionally, responding based on a schedule may be time consuming and expensive.

### SUMMARY

In an aspect, a method includes receiving data characterizing first measurements of water within a water tank recorded by at least one sensor operatively arranged within the water tank. The method further includes determining a first temperature and a first water conductivity level based on the first measurements. The method further includes receiving data characterizing second measurements of the water within the water tank recorded by the at least one sensor. The method further includes determining a second temperature, a second water conductivity level, and a second water level based on the second measurements. The method further includes computing an output data set associated with the water based on the first temperature and the second temperature, the first water conductivity level and the second water conductivity level, and the first water level and the second water level. The method further includes providing the output data set.

One or more of the following features can be included in any feasible combination. For example, the method can include determining a water level within the tank. In some aspects, the method can include determining a first water level based on the first measurements, and determining a second water level based on the second measurements. In other aspects, the output data set can be based on the first water level measurement and the second water level measurement.

In some aspects, the sensor data can include a conductivity level of the water in the water tank over a duration of time. In other aspects, the sensor data can include a temperature level of the water in the water tank over a duration of time. In some aspects, the sensor data can include a water level of the water in the water tank over a duration of time. In other aspects, the output data set can include an alert based on a difference between the first conductivity level and the second conductivity level of the water being outside a threshold range. In certain aspects, the output data set can include information, instructions, and or a control command associated with care of the water tank.

The method can further include activating, based on the output data set, at least one of a heating source, a light source, a water source, or a food source.

In an aspect, a method includes receiving a temperature data set and a water conductivity data set, characterizing measurements of a plurality of characteristic properties of water within a water tank by a sensor operatively arranged within the water tank. The method further includes determining, by a predictive model, a first rule characterizing environmental requirements of the water arranged within the water tank. The method further includes generating an output data set based on the received data sets and the first rule. The method further includes providing the generated output data set.

One or more of the following features can be included in any feasible combination. For example, the method can include receiving a water level data set. In some aspects, the first rule can be based on information associated with the water tank provided by the user, previous measurements of the characteristic properties by the sensor, and the received data sets.

In some aspects, the output data set can include information and/or instructions associated with care of the water tank. In other aspects, the output data set can include a control command associated with stabilization of the water tank. In certain aspects, the control command can be associated with the control at least one of
a heating source, a light source, a water source, or a food source.

In some aspects, the method can further include determining an ammonia level, nitrite level, and/or a nitrate level based on the water conductivity data set.

In some aspects, generating the output data set for the water tank can be further based on one or more of environmental data associated with the water tank, usage of the water tank, location of the water tank, and a type associated with the water tank.

In some aspects, the monitoring system can further include an application on a computing device associated with the user of the water tank, and the receiving of the data sets by the server is via the application. In other aspects, the generated output data set is transmitted to the computing device.

In an aspect, a system includes at least one data processor, and memory storing instructions which, when executed by the at least one data processor, causes the at least one data processor to perform operations including receiving a temperature data set, a water conductivity data set, and a water level data set, characterizing measurements of a plurality of characteristic properties of water within a water tank by a sensor operatively arranged within the water tank. The method further includes determining, by a predictive model, a first rule characterizing environmental requirements of the water arranged within the water tank. The method further includes generating an output data set based on the received data sets and the first rule. The method further includes providing the generated output data set.

One or more of the following features can be included in any feasible combination. For example, the output data set can include at least one of information and/or instructions associated with care of the water tank, an object replenishment recommendation associated with a consumable object within the water tank, or a control command associated with stabilization of the water tank.

In some aspects, the system can further include a Hall Effect sensor, Tunnel Magneto-Resistance (TMR) sensor, or float sensor configured to measure a water conductivity level of water within the water tank.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, and the like.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a flow chart of an exemplary method for providing a recommendation to a target object by an object monitoring system;

FIG. 2 illustrates an exemplary object monitoring system configured to monitor multiple target objects;

FIG. 3 illustrates an exemplary server associated with the object monitoring system of FIG. 2;

FIG. 4 illustrates an exemplary recommendation engine associated with the server of FIG. 3;

FIG. 5 illustrates an exemplary rules engine associated with the server of FIG. 3;

FIG. 6 illustrates an exemplary data processing engine associated with the server of FIG. 3;

FIG. 7 illustrates an exemplary support engine associated with the server of FIG. 3;

FIG. 8 illustrates an exemplary graphical user interface (GUI) display space of an application associated with the object monitoring system of FIG. 2;

FIG. 9 illustrates an exemplary embodiment of a water tank;

FIG. 10A illustrates an exemplary graph of conductivity of water in a tank as a function of time;

FIG. 10B illustrates a graph of time derivative of the conductivity data in FIG. 10A;

FIG. 10C illustrates an exemplary graph of an integral of the derivative data in FIG. 10B;

FIG. 11A illustrates an exemplary graph of conductivity of water in a tank as a function of time; and

FIG. 11B illustrates a graph of time derivative of the conductivity data in FIG. 11A.

### DETAILED DESCRIPTION

Some implementations of the current subject matter can provide automated data outputs during a valuable product's lifecycle by analyzing data about the valuable product's environment, activities, and external information (e.g., time, weather, location, object properties, and the like). Further examples of data can include the end user's experience, preferences, collection, and the like. For example, if an individual has a water tank, such as an enclosure for aquatic organisms, the output data set can include a suggestion for buying a consumable object, such as food, or activating a filter and pump for circulating the water.

Some implementations of the current subject matter can improve the communication characteristics (e.g., communication of recommendations) to ensure effective actions based upon supplier recommendations and end user actions over time. Some implementations of the current subject matter can provide systems and methods that can create opportunities for both the end users and the supplier to form an individual relationship where the end user is connected to the supplier for support while using the product throughout the life cycle of the product. This can allow the supplier to customize the relationship with each individual end user which can help to ensure end user's success with the product. End users can benefit from such a relationship by gaining better results and enjoyment from the products they use, while suppliers can benefit by gaining higher success rates, ratings, repeat sales and ongoing revenue from the products.

In some implementations, an application associated with a computing device of the end user can receive sensor data detected by sensors operatively coupled to the object. The sensor data can include, for example, temperature, humidity, motion, impact, location, sound level, vibration, and the like, associated with the object. This data can be combined with data sourced from outside references such as weather reports, event announcements, emergency incidents, and the like, and can be used by the system to independently assess the state of the object.

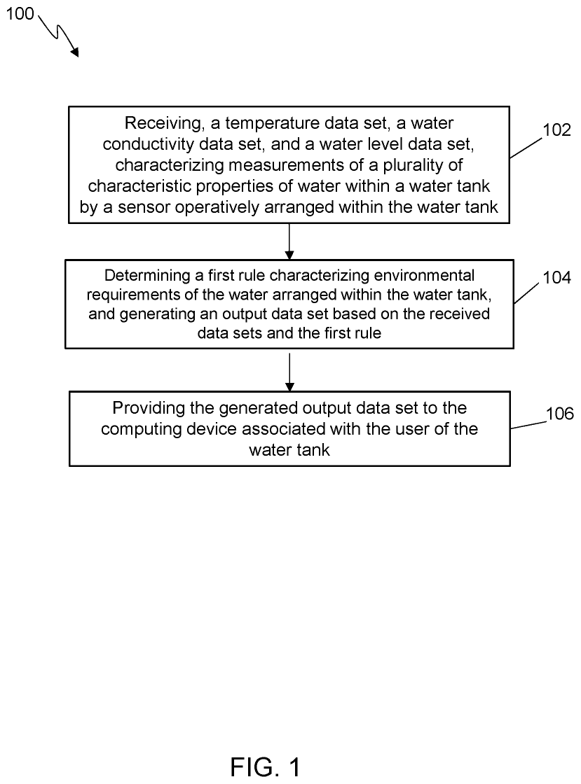

FIG. 1 is a flow chart of an exemplary method for providing an output data set to a water tank by a monitoring system. At 102, data characterizing a plurality of measurements, such as temperature, conductivity, and water level, of a characteristic property of the water tank (e.g., which can be detected by a sensor operatively arranged within the water tank) is received and an output data set relating to the measurements is generated. The data can be received, for example, by a platform (or a server) of the water tank monitoring system.

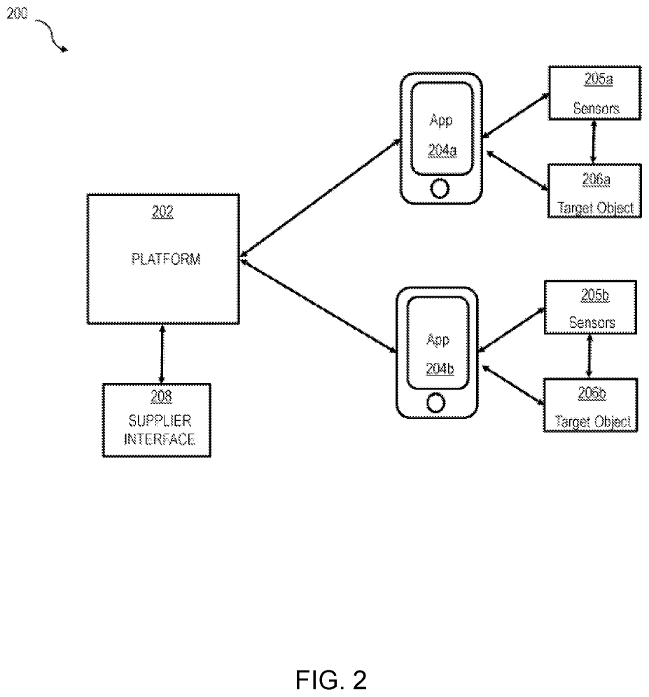

Various components of the water tank monitoring system can be distributed over a cloud, operating devices of users of multiple water tanks, locations of the water tank (e.g., sensors arranged within water tanks and the like). For example, FIG. 2 illustrates an exemplary water tank monitoring system 200 that includes a platform 202; applications 204a and 204b; sensors 205a and 205b; and a supplier interface 208.

The water tank monitoring system 200 can monitor and provide output data sets to the target objects 206a and 206b. The sensor 205a (or 205b) can detect a characteristic property of the target object 206a (or 206b) and transmit the detected characteristic property to the application 204a (or 204b). The application 204a (or 204b) can be installed on a computing device (e.g., laptop, mobile device, and the like) of the user of the target object 206a (or 206b). The application can curate the received sensor data and/or transmit the sensor data to the platform 202. In some implementations, the application can be used by end users to place new orders (e.g., replenishing consumable products), request object information, receive information or instructions to properly maintain the water tank, and the like.

The platform 202 can receive the data from the applications 204a (or 204b) (e.g., data characterizing a measurement of the characteristic property of the target object) and/or sensor data directly from the sensor 205a (or 205b). The supplier interface 208 can allow the supplier to access information in the water tank monitoring system 200 (e.g., information about the product/object, end users, and the like).

Communication among platform 202; applications 204a and 204b; and sensors 205a and 205b can be achieved via one or more of WiFi, Cellular Radio, Bluetooth, low data rate infrastructure, direct wiring, and the like. In some implementations, one or more relay stations can allow for communication among the components of the object monitoring system 200. In some implementations, the various components of the water tank monitoring system 200 can include data storage devices (e.g., memory, RAM, and the like) that can curate received/generated information.

Referring again to FIG. 1, at 104, the platform 202 generates an output data set for the target object 206a (or 206b) based on the received data. As described below, the generation of the recommendation can also be based on various data (e.g., result associated with the implementation of a previous recommendation on the target object 206a, sensor data from multiple target objects, expert data, and the like). Furthermore, the recommendation can be generated by application of various rules (e.g., predetermined rules, rules provided by experts, and the like) on the various data.

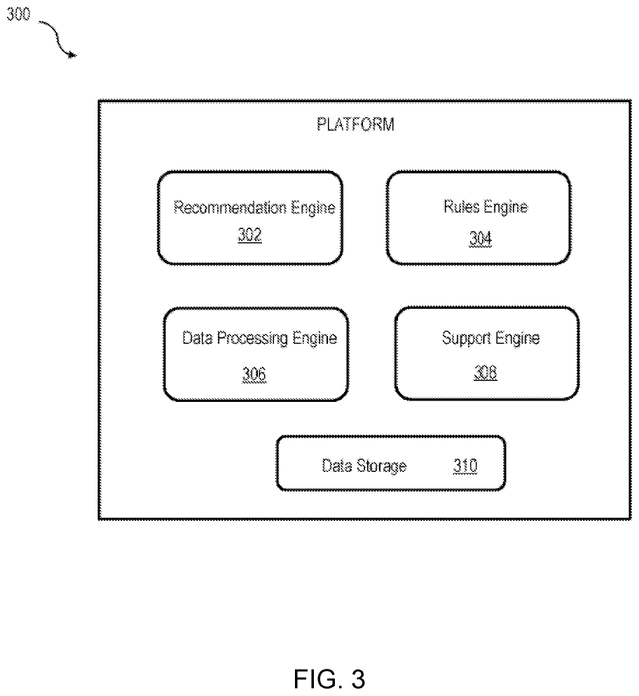

FIG. 3 illustrates an exemplary platform 300. The platform 300 can include a recommendation engine 302, rules engine 304, data processing engine 306, support engine 308 and data storage 310. The platform 300 can receive data from various sources (e.g., sensors operatively coupled to the objects, external database, experts, and the like). The recommendation engine 302 can generate recommendations based on, for example, received data, rules generated by the rules engine 304 (and/or rules from experts). The data processing engine can process the received data (or a portion thereof) and the support engine 308 can respond to queries from the end user.

The data storage 310 can store various information associated with the target objects. For example, data storage 310 can included the lifecycle information of the target object (e.g., stage of the object, milestone of the object, action level criteria, notifications associated with the object, and the like). The data storage 310 can also include information associated with the class or group associated with the target object. The group information can include, for example, summary notice content in target objects of the group, summary notice call to action in multiple target objects in the group, and the like.

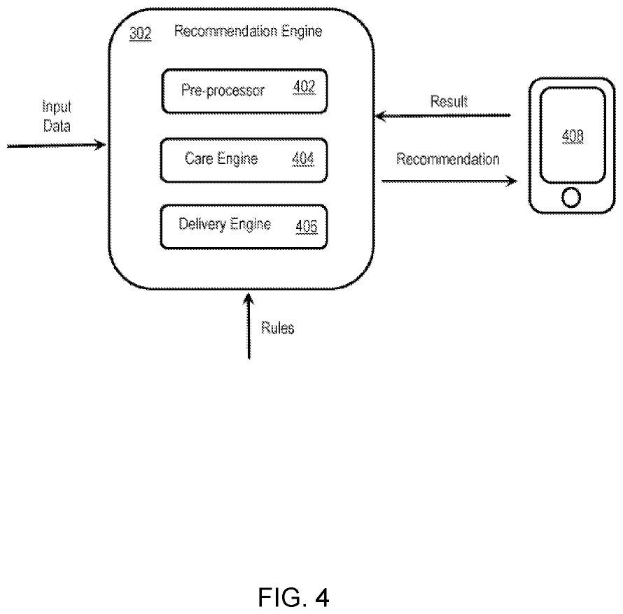

FIG. 4 illustrates an exemplary recommendation engine 302. The recommendation engine 302 can receive data from various sources (e.g., sensor data, rules from the rules engine, data from data storage 310, and the like) and can generate recommendation for the target object. In some implementations, the recommendation can instruct the end user to act to protect, preserve or better use the target object associated with the recommendation. The recommendation engine 302 can include a pre-processor engine 402, a care engine 404 and the delivery engine 406. The pre-processor engine 402 can process received data (e.g., sensor data, data from data storage 310, and the like). In some implementations, the processing of data can be done to prepare (or analyze) the data for execution by the care/delivery engines. For example, received data can be unstructured, or only a fraction of the data may be needed to produce an actionable insight by the care and delivery engines. The pre-processor engine 402 can select the desirable sub-set of data and/or prepare the data for usage by the care/delivery engines.

In some implementations, an algorithm or rule can analyze the received data and determine recommendation characteristics (e.g., whether the recommendation should be in a text form or a video form). This determination can be on historical user response to various recommendations (e.g., how often the user looked at the recommendation, how long the user spent engaging with the object monitoring system, how successful the resulting care actions were, etc.) to determine recommendation characteristics. In some implementations, recommendation characteristics can be transmitted to the delivery engine.

The care engine 404 can receive processed data from the pre-processor 402 and can generate recommendations. For example, the care engine 404 can apply rules (e.g., group rules, individual object rules, expert rules, and the like) received from the rules engine 304 and can apply those rules on the received data. Rules can be applied based on defined triggers. In some implementations, triggers can be time based, based on received data, an external event from a supplier's server, and the like. When a trigger fires, the care engine 404 can determine which rule or rules need to be executed. The execution of the rule may take place on the same server as the care engine or on a different server. The rule may or may not be provided all of the data with the trigger that is needed to execute the rule. If additional data is needed, the care engine 404 may try and get the data from a database, server, or other location. The care engine 404 can process the rule with the limited data, or may stop the execution of the rule.

For example, transmission from a sensor indicating that a water tank, such as a habitat for aquatic animals, has fallen outside of an allowable temperature threshold can trigger the rule engine to determine that a temperature which is too cold corresponds to Rule 1. Rule 1 can state that if the temperature of the water tank is below a certain threshold limit, the user should activate a heat source, or the heat source can be automatically turned on by a processor. Rule 1 may only have the data that the temperature has been below a threshold limit the last 4 hours, so it queries the data from a database to find out if the temperature has been consistently below the threshold limit for 12 hours or not. If the answer is negative, a recommendation can be sent to the owner, or a control command can be sent to activate a heat source. In some exemplary implementations, water level or water conductivity can be measured and compared instead of temperature of the water tank.

In some implementations, the care engine 404 can determine an evaluation parameter for one or more registered target objects by applying the received rules on the received data. Based on the evaluation score, the recommendation engine can make a determination if a recommendation needs to be made. In some implementation, the recommendation can be determined (e.g., selected from a predetermined list of recommendations) based on the evaluation score. The recommendation can indicate, for example, if an operating state (or operating parameter) of the target object should be changed by the end user.

Referring again to FIG. 1, at 106, the generated recommendation (e.g., generated by the care engine 404) can be transmitted to the computing device (e.g., application 204a in the user computing device) associated with the first target object (e.g., 206). The delivery engine 406 can generate parameters associated with the communication (“communication parameters”) of the recommendations generated by the care engine 402. For example, the delivery engine 406 can determine the schedule for providing the recommendation to the user. In some implementations, the communication parameters can be based rules provide by a Digital Marketing Subject Matter Expert (DSME). In some implementations, the delivery engine 406 can determine additional information associated with the recommendation. For example, the delivery engine 406 can determine a schedule associated with the implementation of the recommendation (e.g., when the recommendation needs to be implemented, and the like).

In some implementations, the delivery engine 406 can include a transmission machine learning algorithm. The transmission machine learning algorithm can generate the communication parameters based on information associated with the target object (e.g., personalized information for a given target object or the user of the target object), sensor data from the target object, input from DSME, and the like. In some implementations, the DSME can review and edit the communication parameters.

In some implementations, the recommendation engine 302 can perform multiple iterations (e.g., based on new data, new rules, trigger inputs from the end user, trigger inputs based on predetermined condition, trigger inputs from experts and the like). In some implementations, an input from the PSME can trigger the recommendation engine 302 (e.g., to generate recommendation). The input from the PSME can include state/milestone of the target object, conditions/limiting values associated with the various states of the target object, and the like. The recommendation engine 302 can include one or more of a genetic algorithm, Bayesian network, rete algorithm, inference engine, predictive model, business rule, machine learning model, neural network, classification system (e.g., random forest), regression system (e.g., least squares), and the like. In some implementations, the PSME can instruct the recommendation engine 302 to perform a machine learning process (e.g., based on data in data storage 310).

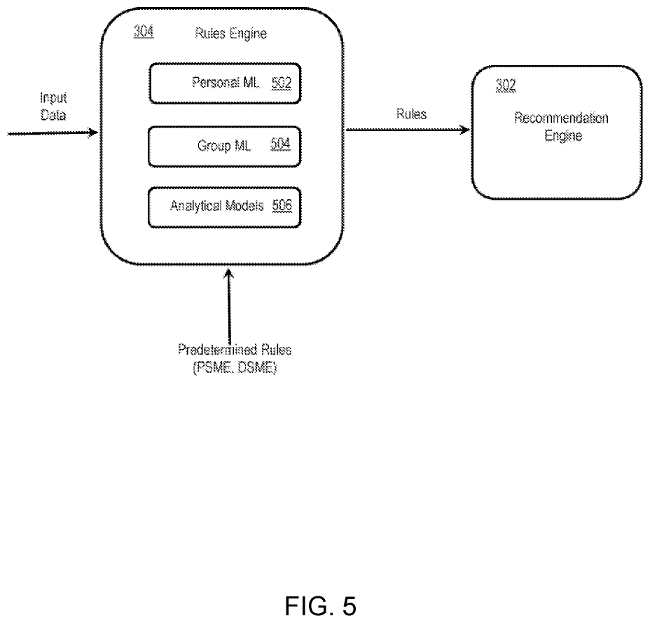

FIG. 5 illustrates an exemplary rules engine 304. The rules engine 304 can include a personal machine learning algorithm 502, a group machine learning algorithm 504 and analytical models 506. The personal machine learning algorithm 502 can generate a first set of object rules based on information associated with a given target object (e.g., personalized information for a given target object or the user of the target object) and/or macro trend information. The target object information can include one or more of data provided the by user of the target object, sensor data from the target object, data associated with a result from the implementation of a previous recommendation (e.g., from the platform 300 to the target object).

The group machine learning algorithm 504 can generate a second set of object rules based on information associated with a group (e.g., predefined group) associated with the target object. For example, the information can include macro trend information associated with the group of target objects. The target object information can include sensor measurements associated with a plurality of target objects in the group, group data from the data storage 310. In some implementations, the information can include personalized information.

In some implementations, generating the first (or second) set of rules can include using predetermined analytical models and varying the properties of the analytical models (e.g., predetermined constants in the analytical model) based on the above-mentioned personalized information (or macro trend information). In some implementations, the analytical models can include previously implemented rules. Based on new information (e.g., newly detected sensor data, new personalized information, new macro trend information, and the like), the previously implemented rules can be modified to generate new rules. In some implementations, the previously implemented first/second set of rules can be modified based on input rules provided by a PSME.

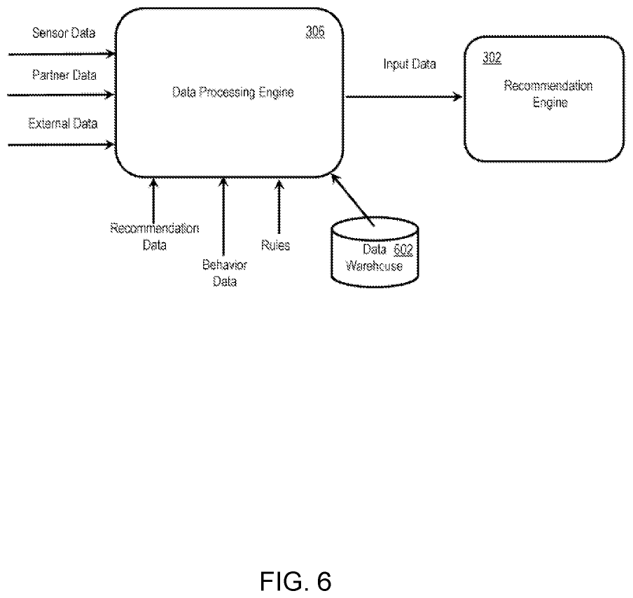

FIG. 6 illustrates an exemplary data processing engine 306. The data processing engine 306 can receive one or more of sensor data, recommendation data (e.g., data characterizing result of implementation of a recommendation), external data (e.g., geographic/weather data associated with a target object), partner data (e.g., data from a partner organization), behavior data (e.g., data associated with the past behavior of the object and/or user of the object), rules (e.g., rules from rules engine 306, and the like). The data processing engine 304 can process the received data to a form that can be used by the recommendation engine 302. In some implementations, the data processing engine can additionally process data and/or transform the data into a form that can be used by other users and services (e.g., supplier servers, data visualization software, data analytics, market research companies, software services such as customer support systems, marketing automation systems, customer relationship management systems, ecommerce systems, content management systems, inventory management, etc.). In some implementations, the data processing engine 306 can receive data from a data warehouse 602 that can store curated data associated with the object monitoring system.

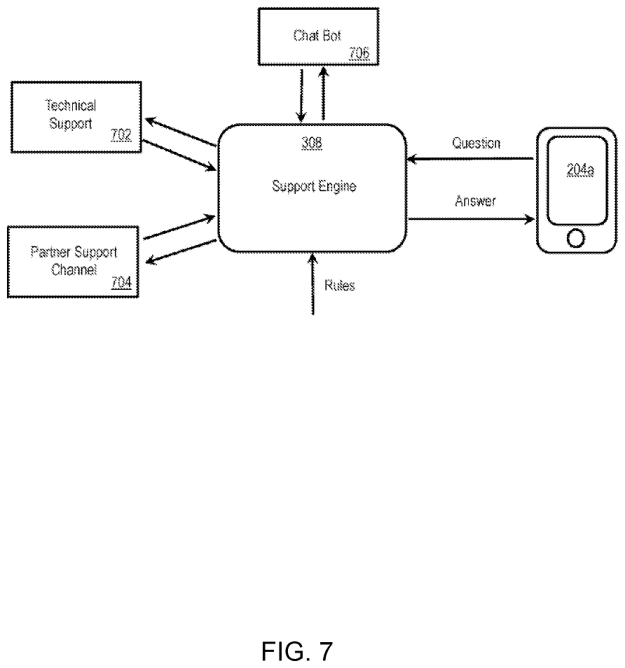

FIG. 7 illustrates an exemplary support engine 308. The support engine 308 can receive question/queries from a user of the application 204a (or 204b) executed in an operating device of the user of the object. For example, the user can ask questions related to the upkeep of the object (e.g., desirable temperature/conductivity associated with the object, precautionary steps that need to be taken in view of an impending set of external conditions, and the like). The support engine 308 can communicate with technical support 702, partner support channel 704, and a chat hot 706 in order to generate a reply to the user question. In some implementations, the support engine can peruse through a predetermined list of question in a database and identify a match for the received question, and generate an answer based on the predetermined list of answers in the database. In some implementations, the support engine 308 can transmit the received question to a technical support 702 or a chat hot 706 and request an answer. In some implementations, the support engine 308 can receive data from a partner support channel 704 and can retrieve the answer from the received data. In some implementations, the support engine 308 can also receive rules (e.g., from PSME, rules engine 304, and the like) and can apply the rules on existing data to determine the answer to the user question. After the answer has been determined the support engine 308 can transmit the answer to the user.

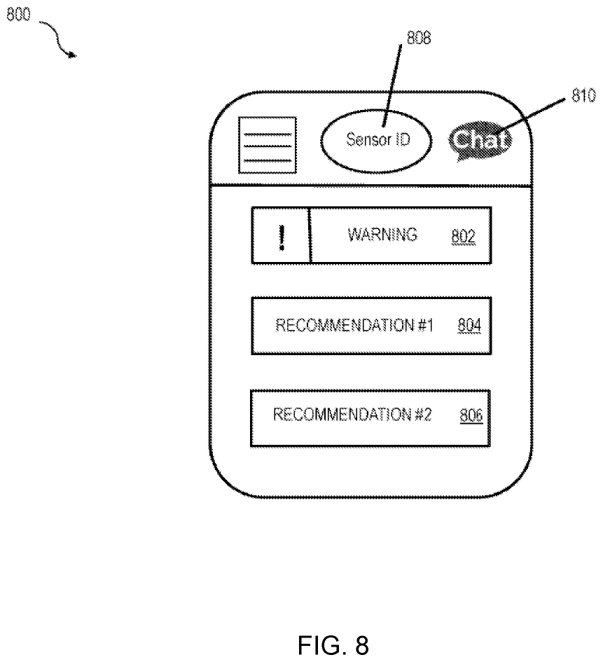

FIG. 8 illustrates an exemplary graphical user interface (GUI) display space 800 associated with the application (e.g., application 204a, 204b, and the like) executed on a user computing device. The GUI display space 800 can include a warning graphical object 802 and recommendation graphical objects 804 and 806.

The warning graphical object 802 can be displayed when the platform 300 transmits a warning signal to the application. For example, based on sensor data, the platform 300 can determine that the operating conditions of the object are undesirable, and can warn the user. In some implementations, the application can include a predetermined set of rules that can be executed by a processor associated with the application. Based on the predetermined set of rules, the application can determine whether the operating conditions (e.g., detected by sensors coupled to the object) are undesirable. Once undesirable operating parameter of the object has been determined, the warning graphical object 802 can be displayed.

The GUI display space 800 can include one or more recommendation graphical objects (e.g., graphical objects 804 and 806). The recommendation graphical objects can include recommendations provided to the application by the platform 300. In some implementations, the recommendation graphical objects can include a schedule for implementations of the recommendation (e.g., several time markers indicative of degree of damage to the water tank and the aquatic animals inside if the recommendation is not implemented). The GUI display space 800 can allow the end user to implement the received recommendation.

The GUI display space 800 can include the identity 806 of the sensor (or the target object) that is under observation by the application. Sensor ID information can be helpful if the application is associated with multiple sensors (or target objects).

The GUI display space 800 can include a query graphical object 810 that can allow the user to interact with the platform 300. For example, the user can transmit questions to the support engine 308 via the query graphical object 810. In some implementations, the end user (or an object associated with the end user) can be registered with the object monitoring system via the GUI display space 800. In some implementations, sensors (e.g., operatively coupled to target objects) can be registered with the object monitoring system via the GUI display space 800.

In view of the above systems, certain water tanks can be monitored using the system as the target object. Some implementations of the current subject matter can include a system to monitor and maintain water tanks including aquatic organisms.

Water tanks (e.g., enclosures of aquatic organisms) may have temperature, water conductivity, and/or water level requirements. For example, it can be desirable to maintain a certain temperature in the water tank (“environment”) and/or maintain the water level in a predetermined range. Additionally or alternately, it can desirable to maintain a certain level of water conductivity in the water tank. This can be achieved, for example, by placing one or more sensors in the water tank that can detect environment conditions (e.g., temperature, conductivity, water level, etc.), and using the detected values to regulate the environment (e.g., by regulating the temperature of heat source, turning on/off a pumps and filters, replacing the light bulbs, etc.). The regulation of various parameters of the environment can include monitoring and maintenance of multiple components (e.g., sensors, lamps, heaters, pumps, filters, etc.) of the water tank. It can be desirable to improve the monitoring system of the environment to detect a current or future need for maintenance of the components in the environment (e.g., change of the water due to high levels of conductivity, clean out of the filter and pump, etc.), and generate an alert signal associated with the maintenance need.

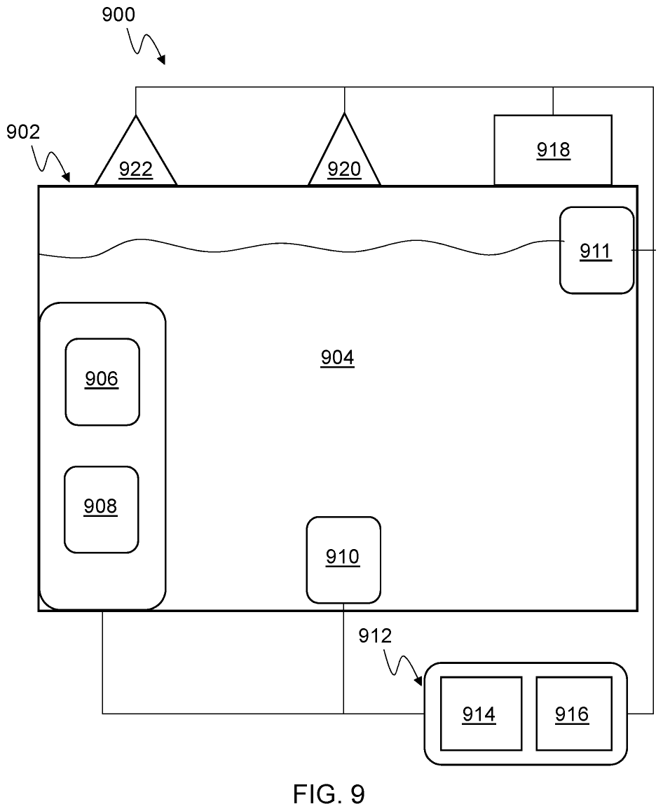

FIG. 9 illustrates a water tank monitoring system 900 which can be used to house aquatic organisms, such as fish, within a water tank 902. As stated above, aquatic organisms can have very specific parameters which must be met within their water tank 902 in order to survive. For example, if the temperature of the water tank 902 is too cold, the aquatic organisms may not be able to digest food properly. Additionally, if a heater is set to a higher than required level, the aquatic organisms can be damaged by the excessive heat of the water 904.

Arranged within the water tank 902 can be water 904. In order to keep the water 904 clean, a pump 906 and a filter 908 can be arranged within the water tank 902 in order to circulate and filter the water 904 within the water tank 902. In order to measure properties of the water 904 within the water tank 902, various sensors 910, 911 can be arranged within the water tank 902. In an exemplary embodiment, the sensors 910 and 911 can be contained within a single housing which is moveable within the water tank 902. Additionally, the control unit 912, including a data processor and a computer readable memory can be communicatively coupled to the sensors 910 and 911 in order to receive measurement data sets related to the properties of the water 904. Also communicatively coupled to the control unit 912 can be dispenser 918, light source 920, and heat source 922. In an exemplary implementation, the dispenser 918 can be configured to dispense food to feed the aquatic organisms within the water tank 902, to dispense chemicals to lower or raise the pH or conductivity of the water 904, and/or to dispense additional water into the water tank 902 if the water level drops below a threshold value.

Sensor 910 can measure the conductivity of the water (e.g., periodically, routinely, etc.). This can be done, for example, by applying an AC signal via a pair of electrodes to the water in the water tank and measuring the voltage drop across the electrodes. In some exemplary implementations, the sensor 910 is a Hall Effect sensor, Tunnel Magneto-Resistance (TMR) sensor, or float sensor. Tracking the trend in conductivity over time can allow for the prediction of various alert conditions before they become critical. For example, as a fish urinates in the tank 902, the ammonia levels increase. Ammonia is an electrolyte which can be measured by an increase in the conductivity of the water. The nitrogen cycle can result in a buildup of nitrates and nitrites in the tank. As one or more of ammonia, nitrite, and nitrate are generated in the tank 902, the conductivity level can increase. Such an increase in conductivity can be indicative of a need for maintenance. Additionally or alternately, a sudden drop in the conductivity can be indicative that maintenance has been performed.

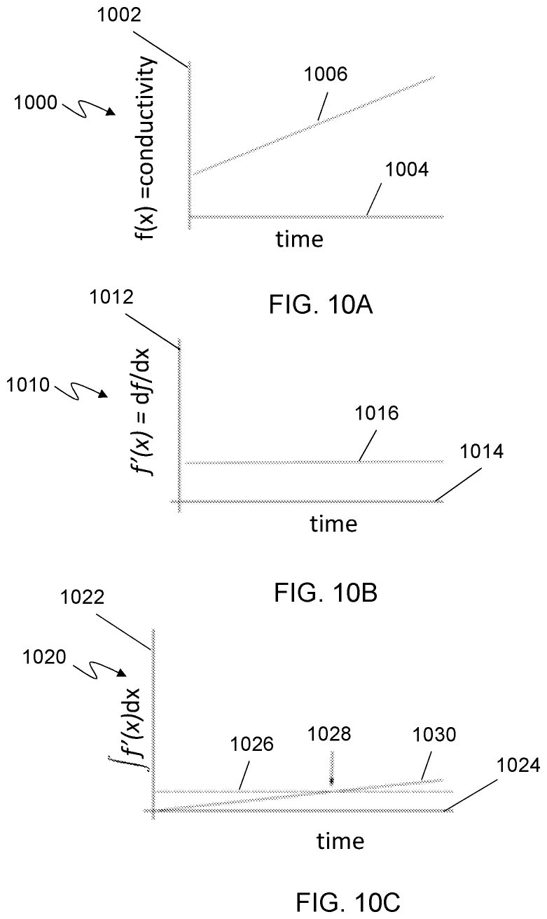

Detecting a change in a property of water 904 (e.g., conductivity) in the water tank 902, and responding to the change (e.g., by changing the water, adding chemical(s) to the water, etc.) in a timely manner can be advantageous. Additionally or alternately, an alert can be generated when the detected change is not attended to in a timely fashion. Once a condition has been identified, ancillary warnings or alerts can be raised. For example, when a conductivity change has been detected a warning/notice to de-chlorinate the water 904 can be generated. By tracking the conductivity over time (e.g., as illustrated in FIG. 10A), complex processing of conductivity measurement (e.g. averaging conductivity over a time window, calculating time derivative/double derivative/integral of conductivity, etc.) can be performed. For example, for a given time-period, the derivative of the conductivity measurement (e.g., as illustrated in FIG. 10B) can be indicative of the frequency, the integral of the frequency (e.g., as illustrated in FIG. 10C) can be indicative of cumulative buildup of electrolytes, etc. When the cumulative buildup of electrolyte (e.g., as calculated by integrating the frequency) is above a certain threshold, an alert can be generated.

FIG. 10A illustrates an exemplary graph 1000 of conductivity of water 204 in the water tank 902 as a function of time. Axis 1002 represents the conductivity of the water 904 as measured by the sensor 910, axis 1004 represents time, and line 1006 represents the determined conductivity of the water 904. As illustrated in the graph 1000, the conductivity 1006 increases over time due to fish urinating in the water, increasing the total dissolved solids (TDS) within the water. If the conductivity becomes too high, the fish within the water tank 902 could be injured or die.

FIG. 10B illustrates a graph 1010 of a time derivative of the conductivity data in FIG. 10A. Axis 1012 represents the derivative of the conductivity of the water 904 as measured by the sensor 910, axis 1014 represents time, and line 1016 represents the derivative of the determined conductivity of the water 904. As illustrated by the line 1016, the rate of change of the conductivity of the water 904 is constant over time.

FIG. 10C illustrates an exemplary graph 1020 of a time integral of the derivative graph 1010 in FIG. 10B. Axis 1022 represents the integral of the derivative of the conductivity of the water 904 as measured by the sensor 910, axis 1024 represents time, and line 1026 represents the integral of the derivative of the determined conductivity of the water 904. As illustrated in FIG. 10C, an alert can be generated when the integral of the first derivative graph 1026 exceeds (or intersects) a predetermined value, represented by line 1026, at point 1028.

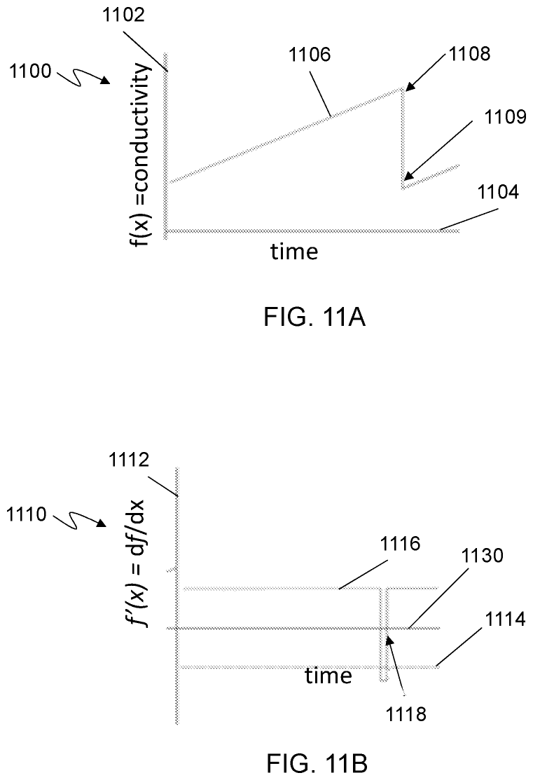

FIG. 11A illustrates an exemplary graph 1100 of conductivity of water 904 in the water tank 902 as a function of time. Axis 1102 represents the conductivity of the water 904 as measured by the sensor 910, axis 1104 represents time, and line 1106 represents the determined conductivity of the water 904. As illustrated in the graph 1100, the conductivity decreases suddenly at point 1108, and begins to increase again at point 1109. This drop in conductivity between point 1108 and 1109 represents an action taken by a user of the monitoring system 900. For example, the user may have changed the water in the tank, replacing the water with a high TDS with a lower average TDS. Alternatively, the monitoring system 900 could have dispensed a chemical agent or additional water to lower the conductivity.

FIG. 11B illustrates a graph 1110 of time derivative of the conductivity data in FIG. 11A. Axis 1112 represents the derivative of the conductivity of the water 904 as measured by the sensor 910, axis 1114 represents time, and line 1116 represents the derivative of the determined conductivity of the water 904. As illustrated in FIG. 11B, an alert can be generated when the plot of the time derivative intersects with a predetermined value, represented by line 1130, at point 1118. As illustrated by the line 1116, the rate of change of the conductivity of the water 904 is constant until point 1118, which corresponds to point 1108 in graph 1100.

The accuracy of alerts can be improved by measuring multiple properties of the water 904 (e.g., temperature, water level, etc.) in addition to conductivity measurement (e.g., as described above). For example, conductivity of the water 904 may vary with temperature, and knowledge of temperature can allow for normalization of conductivity measurement. This can allow for accurate comparison of conductivity of water 904 in the water tank 904 over a period. In some implementations, the temperature of the water tank 902 may need to be maintained within a desired temperature range (e.g., based on the occupants of a tank), and temperature measurements may be used to provide alerts when the temperature of the water tank 902 is outside the desired temperature range. In some implementations, the alert can indicate that the temperature control system needs maintenance.

A water level sensor 911 can be added to improve detection of change of water 904 in the water tank 902. If the water 904 is not regularly changed, the health of the aquatic animals may be at risk. A water level sensor 911 can be based on, for example, Hall effect, TMR (which operates based on the magnetoresistance effect, which refers to a change in resistance induced by the application of an external magnetic field), level switches, or other measurements that can generate a signal representative of the height of the water 904 in the water tank 902. The change in water level due to evaporation may be gradual. A sudden change in water level (e.g., a sudden fall in water level followed by a sudden increase in water level) can indicate that the water 904 in the water tank 902 has been replaced. This information can be cross-referenced with the conductivity signal to increase accuracy of water change detection and/or to identify water changes independent of conductivity measurement. Conditions and alerts can be monitored across multiple water change cycles.

An example of additional tank monitoring can include monitoring a water tank 902 during a process in which the water tank 902 builds up its bio-filter in preparation for adding fish (also referred to as “cycling”). The processes of cycling a tank involves adding nitrifying bacteria to the tank followed by hardy fish, including Poecilids such as the guppy, molly, swordtail and platy, most species of betta, zebra danios, and some tetras, to create ammonia to feed nitrifying bacteria within the water tank 902. As the bacteria grows, it can digest more ammonia and generate nitrites that are converted to nitrates by the filter. The nitrates can be removed through water changes. The rate at which the conductivity increases between water changes can be correlated to the growth of the bio-filter. By monitoring these rates over time, a user can be alerted when their tank is properly “cycled.”

The sensors, processing, and alerting functions can be self-contained in a single unit or can be decoupled. A self-contained unit can have the sensors in the water tank 902 connected to a processor 914 that can sound an alarm when an alert condition has been met. Alternatively, a decoupled unit can have sensors in the water tank 902, which can communicate information associated with the water tank 902 (e.g., conductivity, water level, etc.) wirelessly to a remote server or control unit 912, which can processes the data and generate alerts. The generated alerts can be sent to a mobile phone for display to a user. In the self-contained unit and/or the decoupled sensing unit, the alert system may be connected to a maintenance system that can take remediation actions without user intervention. Examples of remediation actions can include adding fresh water to the tank, dispending food within the tank, activating or deactivating lights based on a day/night cycle, activating a heater, or activating the filter and pump.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof.

These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term, “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term, “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features.

Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases, “at least one of A, B, and C;” “one or more of A, B, and C;” and, “A, B, and/or C” are each intended to mean, “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method, comprising:
receiving data characterizing first measurements of water within a water tank recorded by at least one sensor operatively arranged within the water tank;
determining a first temperature and a first water conductivity level based on the first measurements;
receiving data characterizing second measurements of the water within the water tank recorded by the at least one sensor;
determining a second temperature and a second water conductivity level based on the second measurements;
computing an output data set associated with the water based on the first temperature and the second temperature, and the first water conductivity level and the second water conductivity level; and
providing the output data set.

2. The method of claim 1, wherein the sensor data includes a water level of the water in the water tank.

3. The method of claim 2, the method includes:
determining a first water level based on the first measurements; and
determining a second water level based on the second measurements.

4. The method of claim 3, wherein the output data set is based on the first water level measurement and the second water level measurement.

5. The method of claim 1, wherein the output data set includes an alert based on a difference between the first conductivity level and the second conductivity level of the water being outside a threshold range.

6. The method of claim 1, wherein the output data set includes information, instructions, and/or a control command associated with care of the water tank.

7. The method of claim 6, further comprising activating, based on the control command, at least one of:
a heating source;
a light source;
a water source; or
a food source.

8. A method comprising:
receiving a temperature data set and a water conductivity data set, characterizing measurements of a plurality of characteristic properties of water within a water tank by a sensor operatively arranged within the water tank;
determining, by a predictive model, a first rule characterizing environmental requirements of the water arranged within the water tank;
generating an output data set based on the received data sets and the first rule; and
transmitting the generated output data set.

9. The method of claim 8, further comprising receiving a water level data set.

10. The method of claim 8, wherein the first rule is based on information associated with the water tank provided by the user, previous measurements of the characteristic properties by the sensor, and the received data sets.

11. The method of claim 8, wherein a water level data set characterizes a level of the water in the water tank.

12. The method of claim 8, further comprising determining an ammonia level, nitrite level, and/or a nitrate level based on the water conductivity data set.

13. The method of claim 8, wherein the output data set includes information, instructions, and/or a control command associated with care of the water tank.

14. The method of claim 13, wherein the control command is associated with the control at least one of:
a heating source;
a light source;
a water source; or
a food source.

15. The method of claim 8, wherein generating the output data set for the water tank is further based on one or more of environmental data associated with the water tank, usage of the water tank, location of the water tank, and a type associated with the water tank.

16. The method of claim 8, wherein the monitoring system further includes an application on a computing device associated with the user of the water tank, and the receiving of the data sets by the server is via the application.

17. The method of claim 16, wherein the generated output data set is transmitted to the computing device.

18. A system comprising:
at least one data processor;
memory storing instructions which, when executed by the at least one data processor, causes the at least one data processor to perform operations comprising:
receiving a temperature data set and a water conductivity data set, characterizing measurements of a plurality of characteristic properties of water within a water tank by a sensor operatively arranged within the water tank;
determining, by a predictive model, a first rule characterizing environmental requirements of water arranged within the water tank;
generating an output data set based on the received data sets and the first rule; and
transmitting the generated output data set.

19. The system of claim 18, wherein the output data set includes at least one of:
information and/or instructions associated with care of the water tank;
an object replenishment recommendation associated with a consumable object within the water tank; or
a control command associated with stabilization of the water tank.

20. The system of claim 18, wherein the system further includes a Hall Effect sensor, Tunnel Magneto-Resistance (TMR) sensor, or float sensor configured to measure a water level within the water tank.
